(19) United States
(12) Patent Application Publication
(10) Pub. No.: US 2023/0121208 A1
(43) Pub. Date: Apr. 20, 2023

(54) Dispersible Tablet For Oral Administration

(71) Applicant: Actelion Pharmaceuticals Ltd, Allschwil (CH)

(72) Inventors: Annelies Boiy, Grobbendonk (BE); Ben van Hove, Ranst (BE)

(21) Appl. No.: 18/081,875

(22) Filed: Dec. 15, 2022

Related U.S. Application Data
(63) Continuation of application No. PCT/EP2022/065797, filed on Jun. 10, 2022.

(30) Foreign Application Priority Data
Jun. 11, 2021 (EP) PCT/EP2021/065861

(51) Int. Cl. A61K 31/506; A61K 9/20

(52) U.S. Cl. CPC A61K 31/506; A61K 9/2054; A61K 9/2018; A61K 9/2013

(57) ABSTRACT

The present invention is directed to pharmaceutical compositions or dispersible tablets for oral administration comprising N-[5-(4-bromophenyl)-6-[2-[(5-bromo-2-pyrimidinyl)oxy]ethoxy]-4-pyrimidinyl]-N′-propylsulfamide (macitentan), the use of said pharmaceutical compositions or dispersible tablets for the treatment of pulmonary hypertension and the process for preparing such dispersible tablets.

### CROSS-REFERENCE TO RELATED APPLICATIONS

This application is a continuation of International Patent Application No. PCT/EP2022/065797, filed Jun. 10, 2022, which claims the benefit of the priority of International Patent Application No. PCT/EP2021/065861, filed Jun. 11, 2021, the disclosures of which are incorporated by reference herein.

### FIELD OF THE INVENTION

The present invention is directed to novel pharmaceutical compositions (e.g. dispersible tablets for oral administration) comprising N-[5-(4-bromophenyl)-6-[2-[(5-bromo-2-pyrimidinyl)oxy]ethoxy]-4-pyrimidinyl]-N′-propylsulfamide (also known as macitentan), the use of said pharmaceutical compositions for the treatment of pulmonary hypertension (preferably pulmonary arterial hypertension) or of pulmonary vascular disease and/or cardiac dysfunction in functional single ventricular heart disease patients (especially in Fontan-palliated patients), and processes for preparing said pharmaceutical compositions.

### BACKGROUND OF THE INVENTION

Macitentan is an endothelin receptor inhibitor, useful as an endothelin receptor antagonist. Macitentan and the preparation thereof are described in WO 02/053557. Macitentan has the following structure:

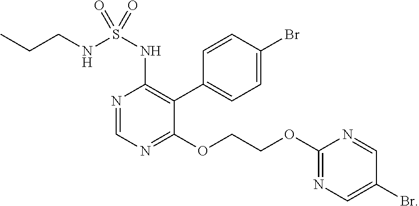 [SMILES: CCCNS(=O)(=O)Nc1ncnc(OCCOc2ncc(Br)cn2)c1-c1ccc(Br)cc1]

Macitentan is an endothelin receptor antagonist that acts as an antagonist of two endothelin (ET) receptor subtypes, ETA and ETB (Kholdani et al, Macitentan for the treatment of pulmonary arterial hypertension. Vasc. Health Risk Manag. (2014), 10, 665-673). Currently, macitentan is taken as a 10 mg oral dose once a day in adult pulmonary arterial hypertension patients.

Previous formulations of macitentan pediatric dispersible tablets have been studied and shown to be biocomparable to adult film-coated tablet formulations and well tolerated (Sidharta et al., Pharmacol. Res. Perspect., 2020, 1-8). These pediatric macitentan dispersible tablets have an inner phase containing 0.5, 2.5 or 5 mg of macitentan, delta-mannitol, croscarmellose sodium and isomalt, and an outer phase comprising mannitol, croscarmellose sodium, isomalt and magnesium stearate (Table 1) and were prepared according to a process summarized by the flow chart shown in FIG. 1 (wet granulation).

TABLE 1
Composition of the 0.5 mg, 2.5 mg and 5.0 mg bi-phasic Macitentan
oral dispersible tablets prepared by wet granulation.

| | | Quantity per Unit (mg) | | |
|---|---|---|---|---|
| | | 0.5 mg | 2.5 mg | 5.0 mg |
| Component | Function | tablets | tablets | tablets |

Intragranular Phase

| | | | | |
|---|---|---|---|---|
| Macitentan | Active Ingredient | 0.5 | 2.5 | 5.0 |
| Delta-Mannitol | Filler | 29.5 | 27.5 | 25.0 |
| Isomalt | Binder | 2.5 | 2.5 | 2.5 |
| Sodium | Disintegrant | 2.5 | 2.5 | 2.5 |
| Croscarmellose | | | | |
| Purified Water ^{a} | Solvent | q.s. | q.s. | q.s. |

Extragranular Phase

| | | | | |
|---|---|---|---|---|
| Parteck ® ODT | Filler/Disintegrant | 9.5 | 9.5 | 9.5 |
| Isomalt | Binder/ | 2.5 | 2.5 | 2.5 |
| | compressibility | | | |
| | enhancer | | | |
| Sodium | Disintegrant | 2.5 | 2.5 | 2.5 |
| Croscarmellose | | | | |
| Magnesium Stearate | Lubricant | 0.5 | 0.5 | 0.5 |

| | | | |
|---|---|---|---|
| Tablet Weight: | 50.0 | 50.0 | 50.0 |

^{a} Removed during processing
^{b} Parteck ® ODT (orally disintegrating tablet) is a combination of specifically spray-granulated mannitol and croscarmellose sodium.

The development of tablets of potent drugs poses several challenges, including the insurance of a homogenous distribution of active pharmaceutical ingredient (API) is of concern due to inadequate uniformity could result in failure of therapy. The difficulty of distributing a small amount of API evenly into a large mass of excipients is a commonly recognized technical challenge. For this reason, typically low dose solids are often manufactured through a wet granulation process (Table 1, FIG. 1).

Technical challenges with respect to compressibility and type of excipients may however arise, limiting the robustness of the wet granulation process. Thus, in the specific case of macitentan, there was the need to design particular pharmaceutical compositions or dispersible tablets comprising macitentan fulfilling special technical requirements and overcoming the previously mentioned technical challenges. To overcome these challenges, direct compression (DC) process was explored to optimize the manufacturing process and the specific compositions conceived for that purpose surprisingly exhibited improved uniformity for homogenous distribution of API.

Direct Compression (DC) is the most straightforward manufacturing option, with the fewest manufacturing steps, making it the easiest to control and least expensive. The DC tablet manufacturing process uses two primary process steps: blending the API with excipients and compressing the finished tablets. Because of the simplicity of the process, the DC process is directly impacted by material properties. DC requires increased performance, quality, and consistency from starting ingredients, including excipients. As δ-mannitol is specifically designed for wet granulation, conversion to a DC process implicates the need for change to a β-mannitol. The main drawback of the commonly used β polymorph of mannitol in tablet formulations is its low compressibility.

### SUMMARY OF THE INVENTION

The present invention is directed to pharmaceutical compositions (e.g. dispersible compositions) comprising macitentan or a pharmaceutically acceptable salt, solvate, hydrate or morphological form thereof; methods of preparation of said pharmaceutical compositions; and method of treating pulmonary hypertension (preferably pulmonary arterial hypertension), or of treating pulmonary vascular disease and/or cardiac dysfunction in functional single ventricular heart disease patients (especially in Fontan-palliated patients), comprising administration of said pharmaceutical compositions.

Within the context of this disclosure, any reference to macitentan is to be understood as referring also to the pharmaceutically acceptable salts or solvates, including hydrates as well as to the morphological forms thereof, if not indicated otherwise and where appropriate and expedient.

In certain embodiments, the present invention is directed to a pharmaceutical composition comprising macitentan, and β-mannitol; wherein said pharmaceutical composition exhibits improved AP content uniformity, homogenous distribution of API with improved process for ease of making, particularly at commercial scale.

An embodiment of the present invention is directed to a pharmaceutical composition or dispersible tablet comprising:

a. macitentan,

b. β-mannitol,

c. isomalt

d. croscarmellose sodium, and

e. magnesium stearate.

In another embodiment of the present invention is directed to a pharmaceutical composition or dispersible tablet comprising:

a. about 0.5-20% w/w macitentan,

b. about 0.1-90% w/w mannitol,

c. about 0.1-90% w/w of isomalt,

d. about 5-20% w/w of croscarmellose sodium, and

e. about 0.5-5% w/w of magnesium stearate.

In another embodiment of the present invention is directed to a pharmaceutical composition or dispersible tablet comprising:

a. about 0.5-5% w/w macitentan,

b. about 0.1-90% w/w mannitol,

c. about 0.1-90% w/w of isomalt,

d. about 5-20% w/w of croscarmellose sodium, and

e. about 0.5-5% w/w of magnesium stearate.

In a further embodiment of the present invention is directed to a pharmaceutical composition or dispersible tablet comprising:

a. about 1% w/w macitentan,

b. about 75% w/w mannitol,

c. about 10% w/w of isomalt,

d. about 11% w/w of croscarmellose sodium, and

e. about 3% w/w of magnesium stearate.

In various embodiments, the present invention is directed to a pharmaceutical composition comprising macitentan. In various embodiments, the pharmaceutical composition comprises macitentan and β-mannitol. In various embodiments, the pharmaceutical composition comprises macitentan, β-mannitol, and one or more of isomalt, croscarmellose sodium, and magnesium stearate.

In an embodiment, the present invention is directed to a dispersible tablet comprising macitentan. In various embodiments, the dispersible tablet comprises macitentan and β-mannitol. In various embodiments, the dispersible tablet includes macitentan, β-mannitol, and one or more of isomalt, croscarmellose sodium, and magnesium stearate.

In another embodiment, the present invention is directed to a method of preparing a pharmaceutical composition comprising macitentan. In various embodiments, the present invention is directed to a method or preparing a pharmaceutical composition comprises macitentan and β-mannitol. In various embodiments, the present invention is directed to a method of preparing a pharmaceutical composition comprising macitentan, β-mannitol, and one or more of isomalt, croscarmellose sodium, and magnesium stearate.

In another embodiment, the present invention is directed to a method for preparing dispersible tablets comprising macitentan. In various embodiments, the present invention is directed to a method for preparing dispersible tablets comprise macitentan and β-mannitol. In various embodiments, the present invention is directed to a method for preparing dispersible tablets comprise macitentan, β-mannitol, isomalt, croscarmellose sodium, and magnesium stearate.

In an embodiment, the instant invention is directed to a method of treating pulmonary arterial hypertension by administering to a patient in need thereof a pharmaceutical composition as described herein.

In an embodiment, the instant invention is directed to a method of treating pulmonary arterial hypertension by administering to a patient in need thereof a pharmaceutical composition comprising macitentan and β-mannitol.

In an embodiment, the instant invention is directed to a method of treating pulmonary arterial hypertension by administering to a patient in need thereof a pharmaceutical composition comprising macitentan, β-mannitol, and one or more of isomalt, croscarmellose sodium, and magnesium stearate. In an embodiment, the instant invention is directed to a method of treating pulmonary arterial hypertension comprising administering to a patient in need thereof a pharmaceutical composition comprising macitentan, β-mannitol, isomalt, croscarmellose sodium, and magnesium stearate.

In an embodiment, the instant invention is directed to a method of treating pulmonary arterial hypertension comprising administering to a patient in need thereof a dispersible tablet as described herein. In an embodiment, the instant invention is directed to a method of treating pulmonary arterial hypertension comprising administering to a patient in need thereof a dispersible tablet comprising macitentan and β-mannitol. In an embodiment, the instant invention is directed to a method of treating pulmonary arterial hypertension comprising administering to a patient in need thereof a dispersible tablet comprising macitentan, β-mannitol, and one or more of isomalt, croscarmellose sodium, and magnesium stearate. In an embodiment, the instant invention is directed to a method of treating pulmonary arterial hypertension comprising administering to a patient in need thereof a dispersible tablet comprising macitentan, β-mannitol, isomalt, croscarmellose sodium, and magnesium stearate.

In certain embodiments of the instant invention, the pulmonary hypertension is pulmonary arterial hypertension.

In particular, the pulmonary hypertension or pulmonary arterial hypertension patients to whom the previously described methods of treatment are addressed will be pediatric patients, i.e. patients aged 18 years or less.

In another embodiment, the instant invention is directed to a method of treating pulmonary vascular disease and/or cardiac dysfunction in a Functional Single Ventricular Heart Disease (FVSHD) patient comprising administering to said patient in need thereof a pharmaceutical composition as described herein.

In an embodiment, the instant invention is directed to a method of treating pulmonary vascular disease and/or cardiac dysfunction in a FSVHD patient comprising administering to said patient in need thereof a pharmaceutical composition comprising macitentan and β-mannitol.

In an embodiment, the instant invention is directed to a method of treating pulmonary vascular disease and/or cardiac dysfunction in a FSVHD patient comprising administering to said patient in need thereof a pharmaceutical composition comprising macitentan, β-mannitol, and one or more of isomalt, croscarmellose sodium, and magnesium stearate. In an embodiment, the instant invention is directed to a method of treating pulmonary vascular disease and/or cardiac dysfunction in a FSVHD patient comprising administering to said patient in need thereof a pharmaceutical composition comprising macitentan, β-mannitol, isomalt, croscarmellose sodium, and magnesium stearate.

In an embodiment, the instant invention is directed to a method of treating pulmonary vascular disease and/or cardiac dysfunction in a FSVHD patient comprising administering to said patient in need thereof a dispersible tablet as described herein. In an embodiment, the instant invention is directed to a method of treating pulmonary arterial hypertension comprising administering to a patient in need thereof a dispersible tablet comprising macitentan and β-mannitol. In an embodiment, the instant invention is directed to a method of treating pulmonary vascular disease and/or cardiac dysfunction in a FSVHD patient comprising administering to said patient in need thereof a dispersible tablet comprising macitentan, β-mannitol, and one or more of isomalt, croscarmellose sodium, and magnesium stearate. In an embodiment, the instant invention is directed to a method of treating pulmonary vascular disease and/or cardiac dysfunction in a FSVHD patient comprising administering to said patient in need thereof a dispersible tablet comprising macitentan, β-mannitol, isomalt, croscarmellose sodium, and magnesium stearate.

In certain embodiments of the instant invention, the FSVHD patients in whom the pulmonary vascular disease and/or cardiac dysfunction is treated are Fontan-palliated patients.

In particular, the FSVHD patients to whom the previously described methods of treatment are addressed will be pediatric patients, i.e. patients aged 18 years or less.

### BRIEF DESCRIPTION OF THE FIGURES

FIG. 1 shows a flowchart of Batch Manufacturing Process of the 0.5 mg, 2.5 mg and 5.0 mg oral dispersible tablets by wet granulation.

FIG. 2 shows a flowchart of Batch Manufacturing Process for the 1 mg and 2.5 mg oral dispersible tablet by direct compression according to the instant invention.

FIG. 3 shows the stability profile for the different grades of β-mannitol excipients used in the manufacturing process. This figure shows the amount of 6-amino-5-(4-bromophenyl)-5-(2-((5-bromopyrimidin-2-yl)oxy)ethoxy)-pyrimidine (“Compound A”—hydrolysis degradation product of macitentan) within the tablet after storage conditions 50° C./10% relative humidity (RH) (short bar) and 50° C./75% RH in comparison with the initial profile (tall bar).

FIG. 4a shows the compression force-hardness profile comparison of the wet granulation tablets versus the direct compression tablets. A higher tablet hardness is achieved at lower compression forces for the DC formula compression force-hardness profile

FIG. 4b shows the compression force-disintegration time profile comparison of the wet granulation tablets versus the direct compression tablets.

FIG. 4c shows the dissolution profile of the wet granulation vs. direct compression formulations.

FIG. 5 shows the dissolution profile comparison of macitentan dispersible tablets containing 1 up to 3% magnesium stearate. An increase in magnesium stearate up to 3% did not impact on the dissolution of the macitentan dispersible tablets.

FIG. 6 shows the dissolution profiles for three different macitentan dispersible tablet dose strengths: 1 mg, 2.5 mg, and 3.5 mg.

### DETAILED DESCRIPTION

The present invention is directed to pharmaceutical compositions or dispersible tablets comprising macitentan or a pharmaceutically acceptable salt, solvate, hydrate or morphological form thereof; methods of preparation of said pharmaceutical compositions; and methods of treating pulmonary hypertension or of treating pulmonary vascular disease and/or cardiac dysfunction in functional single ventricular heart disease patients (especially in Fontan-palliated patients), comprising administration of said pharmaceutical compositions or dispersible tablets.

One skilled in the art will recognize that wherein the term “macitentan” is used in the description of an embodiment of the present invention, it is intended that said term include macitentan as well as pharmaceutically acceptable salts, solvates, hydrates and morphological forms thereof.

The present invention is directed to a pharmaceutical composition comprising macitentan, and β-mannitol. In various embodiments, the present invention is directed to a pharmaceutical composition comprising macitentan, β-mannitol, and one or more of isomalt, croscarmellose sodium, and magnesium stearate.

In various embodiments, the present invention is directed to pharmaceutical compositions wherein macitentan is present in an amount in the range from about 0.5 mg to about 10 mg, or any amount or range thereof; more preferably in an amount in the range of from about 1 mg to about 5 mg, or any amount or range thereof. In various embodiments, the present invention is directed to pharmaceutical compositions wherein macitentan is present in an amount of about 1 mg, about 2.5 mg, about 3.5 mg or about 5.0 mg.

In various embodiments, the present invention is directed to a pharmaceutical composition comprising the amount of macitentan may vary from 0.5-20% w/w, and the amount of β-mannitol may vary from 0.1-90% w/w. In various embodiments, the instant invention is directed to a pharmaceutical composition comprising macitentan in an amount that may vary from 1% w/w, β-mannitol in an amount that may vary from 0.1-90% w/w, and one or more of isomalt in an amount that may vary from 0.1-90% w/w, croscarmellose sodium in an amount that may vary from 5-20% w/w and magnesium stearate in an amount that may vary from 0.5-5% w/w.

In various embodiments, the present invention is directed to a pharmaceutical composition comprising the amount of macitentan may vary from 0.5-5% w/w, and the amount of β-mannitol may vary from 0.1-90% w/w. In various embodiments, the instant invention is directed to a pharmaceutical composition comprising macitentan in an amount that may vary from 0.5-5% w/w, β-mannitol in an amount that may vary from 0.1-90% w/w, and one or more of isomalt in an amount that may vary from 0.1-90% w/w, croscarmellose sodium in an amount that may vary from 5-20% w/w and magnesium stearate in an amount that may vary from 0.5-5% w/w.

In an embodiment, the present invention is notably directed to a pharmaceutical composition comprising macitentan in an amount of 0.9-1.1% w/w, β-mannitol in an amount of 67.5-82.5% w/w, isomalt in an amount of 9-11% w/w, croscarmellose sodium in an amount of 9.9-12.1% w/w and magnesium stearate is present in an amount of about 2.7-3.3% w/w, for example a pharmaceutical composition comprising macitentan in an amount of 1% w/w, β-mannitol in an amount of 75% w/w, isomalt in an amount of 10% w/w, croscarmellose sodium in an amount of 11% w/w and magnesium stearate is present in an amount of 3% w/w.

In an embodiment, the instant invention is directed to a pharmaceutical composition comprising macitentan in an amount of about 1 w/w, β-mannitol in an amount of about 75% w/w, isomalt in an amount of about 10% w/w, croscarmellose sodium in an amount of about 11% w/w and magnesium stearate is present in an amount of about 3% w/w.

The present invention is directed to a dispersible tablet comprising macitentan, and β-mannitol. In various embodiments, the present invention is directed to a dispersible tablet comprising macitentan, β-mannitol, and one or more of isomalt, croscarmellose sodium, and magnesium stearate.

In various embodiments, the present invention is directed to a dispersible tablet comprising macitentan present in an amount in the range from about 0.5 mg to about 10 mg, or any amount or range thereof; more preferably in an amount in the range of from about 1 mg to about 5 mg, or any amount or range thereof. In various embodiments, the present invention is directed to dispersible tablet wherein macitentan is present in an amount of about 1 mg, about 2.5 mg, about 3.5 mg or about 5.0 mg.

In another embodiment, the present invention is directed to a dispersible tablet comprising macitentan in the amount that may vary from 0.5-20% w/w, and the amount of β-mannitol may vary from 0.1-90% w/w. In various embodiments, the instant invention is directed to a dispersible tablet comprising macitentan in an amount that may vary from 0.5-20% w/w, β-mannitol in an amount that may vary from 0.1-90% w/w, and one or more of isomalt in an amount that may vary from 0.1-90% w/w, croscarmellose sodium in an amount that may vary from 5-20% w/w and magnesium stearate in an amount that may vary from 0.5-5% w/w.

In various embodiments, the present invention is directed to a dispersible tablet comprising macitentan in the amount that may vary from 0.5-5% w/w, and the amount of β-mannitol may vary from 0.1-90% w/w. In various embodiments, the instant invention is directed to a dispersible tablet comprising macitentan in an amount that may vary from 0.5-5% w/w, β-mannitol in an amount that may vary from 0.1-90% w/w, and one or more of isomalt in an amount that may vary from 0.1-90% w/w, croscarmellose sodium in an amount that may vary from 5-20% w/w and magnesium stearate in an amount that may vary from 0.5-5% w/w.

In an embodiment, the present invention is notably directed to a dispersible tablet comprising 0.9-1.1% w/w macitentan, 67.5-82.5% w/w β-mannitol, 9-11% w/w of isomalt, 9.9-12.1% w/w of croscarmellose sodium, and 2.7-3.3% w/w of magnesium stearate, for example a dispersible tablet comprising 1% w/w macitentan, 75% w/w β-mannitol, 10% w/w of isomalt, 11 w/w of croscarmellose sodium, and 3% w/w of magnesium stearate.

In an embodiment, the instant invention is directed to a dispersible tablet comprising macitentan in an amount of about 1% w/w, β-mannitol in an amount of about 75% w/w, isomalt in an amount of about 10% w/w, croscarmellose sodium in an amount of about 11% w/w and magnesium stearate is present in an amount of about 3% w/w.

In various embodiments, macitentan is present in an amount of about 0.5-20% w/w.

In various embodiments, macitentan is present in an amount of about 1 mg, about 2.5 mg, about 3.5 mg or about 5.0 mg in the pharmaceutical composition or dispersible tablet.

In an embodiment, the instant invention wherein the mannitol is β-mannitol.

In an embodiment, the instant invention is directed to a pharmaceutical composition, preferably a dispersible tablet, wherein the β-mannitol grade is selected from Table 2 (below):

TABLE 2
Grades of β-mannitol

| | Particle size | | |
|---|---|---|---|
| Mannitol grade | distribution | Specific surface area | |
| Parteck ® M200 | D50 142-231 μm | 3.0 | m²/g [Merck] |
| Pearlitol ® SD100 | D10-50-90: | 1.4 | m²/g [PreTaP] |
| | 64-111-165 μm | 0.6 | m²/g [Roquette] |
| Parteck ® ODT | D50 70-120 μm | 2.4-3.5 | m²/g [Merck] |

In another embodiment, the instant invention is directed to a pharmaceutical composition, preferably a dispersible tablet, wherein the β-mannitol has a particle size distribution (PSD) such that, when measured according to the method entitled “Laser diffraction method for determining Particle Size Distribution” described in the “Methods” section below, the D10 value is from 10 to 60 μm, the D50 value is from 60 to 140 and the D90 value is from 140 to 220 μm. In particular, the β-mannitol used will have a maximum water content of 3%.

In another embodiment, the instant invention is directed to a pharmaceutical composition, preferably a dispersible tablet, wherein the β-mannitol has a specific surface area (SSA) of 2 m²/g or less, and preferably of 0.5 to 1.5 m²/g when measured according to the method entitled “BET method for determining Specific Surface Area” described in the “Methods” section below. In a further embodiment, the instant invention is directed to a pharmaceutical composition, preferably a dispersible tablet, wherein the β-mannitol has a PSD such that, when measured according to the method entitled “Laser diffraction method for determining Particle Size Distribution” described in the “Methods” section below, the D10 value is from 10 to 60 μm, the D50 value is from 60 to 140 and the D90 value is from 140 to 220 μm, and wherein the β-mannitol has a SSA of 2 m²/g or less, and preferably of 0.5 to 1.5 m²/g when measured according to the method entitled “BET method for determining Specific Surface Area” described in the “Methods” section below. In particular, the β-mannitol used will have a maximum water content of 3%.

In another embodiment, the instant invention is directed to a pharmaceutical composition, preferably a dispersible tablet, comprising isomalt, wherein the isomalt wherein the PSD is such that the D90 is less than about 360 μm and the isomalt has a solubility of 42 g per 100 g solution in 20° C. water.

In an embodiment, the instant invention is directed to a pharmaceutical composition, preferably a dispersible tablet, wherein β-mannitol is present in an amount of about 0.1-90% w/w.

In an embodiment, the instant invention is directed to a pharmaceutical composition, preferably a dispersible tablet, wherein isomalt is present in an amount of about 0.1-90% w/w.

In an embodiment, the instant invention is directed to a pharmaceutical composition, preferably a dispersible tablet, wherein croscarmellose sodium is present in an amount of about 5-20% w/w.

In an embodiment, the instant invention is directed to a pharmaceutical composition, preferably a dispersible tablet, wherein magnesium stearate is present in an amount of about 0.5-5% w/w.

In various embodiments, the instant invention is directed to a pharmaceutical composition, preferably a dispersible tablet, comprising macitentan in an amount of about 1% w/w, β-mannitol in an amount of about 75% w/w, and one or more of isomalt in an amount of about 10% w/w, croscarmellose sodium in an amount of about 11% w/w and magnesium stearate in an amount of about 3% w/w.

In another embodiment, the pharmaceutical composition of the instant invention is in the form of a dispersible tablet.

In another embodiment, the dispersible tablet of the invention has a hardness of 20 to 120 N.

In another embodiment, the instant invention is directed to a method of treating pulmonary hypertension comprising administering to a patient in need thereof a macitentan pharmaceutical composition comprising macitentan, and β-mannitol.

In another embodiment, the instant invention is directed to a method of treating pulmonary hypertension comprising administering to a patient in need thereof a macitentan pharmaceutical composition comprising macitentan, and β-mannitol, and one or more of isomalt, croscarmellose sodium, and magnesium stearate.

In another embodiment, the instant invention is directed to a method of treating pulmonary hypertension, wherein the pulmonary hypertension is pulmonary arterial hypertension.

The formulations herein may be prepared by dry blending and compression into dispersible/chewable/swallowable/quick dissolving tablets as described notably in Lieberman, Lachman & Schwarz, “Pharmaceutical Dosage Forms: Tablets” (1989). In one embodiment, the instant invention relates to a dispersible tablet as described above having a total weight of 500 mg or less which, when placed in a tablespoon containing 2-5 ml of water, preferably 3 ml of water, fully disperses in 5 min or less (and preferably in 2 min or less and more preferably in 1 min or less) when tested according to the method entitled “Disintegration on a spoon” described in the “Methods” section below.

The pharmaceutical compositions or dispersible tablets according to the invention may be used as medicaments.

The pharmaceutical compositions or dispersible tablets may be used for the preparation of medicaments for use in the treatment of pulmonary hypertension, in particular pulmonary arterial hypertension, or for use in the treatment of pulmonary vascular disease and/or cardiac dysfunction in functional single ventricular heart disease patients (especially in Fontan-palliated patients).

Pulmonary hypertension (PH) was reported for the first time in 1891 when the autopsy of a patient with sudden death revealed right ventricular hypertrophy and pulmonary artery sclerosis without any apparent cause. Pulmonary arterial hypertension (PAH) is a subgroup of PH and it is a progressive disease with elevated pulmonary vascular resistance (PVR) as the basic cause for increased right ventricular afterload and hypertrophy, which eventually proceeds to right ventricular dilatation and failure, and premature death. PH is clinically classified into five groups according to the World Health Organization (WHO) classification: pulmonary arterial hypertension (PAH) (group 1), PH related to left heart disease (group 2), PH due to lung disease and/or hypoxia (group 3), chronic thromboembolic PH and other pulmonary artery obstructions (group 4), and PH with unclear and/or multifactorial mechanisms (group 5) (Roger Hullin, Cardiovascular Medicine (2018), 21(7-8):195-199; Simonneau et al., Haemodynamic definitions and updated clinical classification of pulmonary hypertension. Eur. Respir. J. (2018), Dec. 13. pii: 1801913. doi: 10.1183/13993003.01913-2018. [Epub ahead of print]).

Concerning PAH, the present invention focuses on PAH which is haemodynamically characterized by the presence of a mean pulmonary artery pressure (PAP)>20 mm Hg, a pulmonary artery wedge pressure (PAWP)≤15 mm Hg and a PVR of equal to or more than (>) 3 Wood units, alternatively >2 Wood units, all measured at rest. In particular, the present invention focuses on PAH which is haemodynamically characterized by the presence of a mean pulmonary artery pressure (PAP)≥25 mm Hg, a pulmonary artery wedge pressure (PAWP)≤15 mm Hg and a PVR of equal to or more than (>) 3 Wood units, alternatively >2 Wood units, all measured at rest.

Functional single ventricular heart disease patients include both patients with hearts with only one ventricle and patients with hearts with two ventricles that are not amenable to biventricular repair (see Frescura and Thiene G. (2014), Front Pediatr. (2014), 2, 62. The new concept of univentricular heart). Functional single ventricular heart disease patients include Fontan-palliated patients, that is, patients, typically children, with univentricular hearts or other related congenital heart diseases patients which have undergone the so-called Fontan procedure. The latter is a palliative surgical procedure that involves diverting the venous blood from the inferior vena cava (IVC) and superior vena cava (SVC) to the pulmonary arteries without passing through the morphologic right ventricle, i.e., the systemic and pulmonary circulations are placed in series with the functional single ventricle. The procedure was initially performed 1968 by Francis Fontan and Eugene Baudet. Contemporary modifications of surgical techniques have significantly improved survival. Overall, the Fontan patients have however decreased long-term survival, progressively deteriorating functional status and an increased risk of sudden death, which means that the procedure is considered to be palliative rather than curative (Fontan et al., Circulation (1990), 81, 1520-1536).

Many post-Fontan complications arise as a result of increased venous pressure and congestion, and chronic low blood flow/cardiac output. Given the lack of ventricular force to drive blood flow through the pulmonary arteries, a low resistance (pulmonary vascular resistance [PVR]) and high capacitance system are mandatory for a well-functioning Fontan circuit. In the postoperative period, even small increases in PVR can lead to systemic venous hypertension associated with a decreased cardiac output despite a technically successful operation (Kirklin et al., Eur. J. Cardiothorac. Surg. (1990), 4, 2-7). In fact, a slight increase in PVR at any time to levels that would be readily tolerated in normal physiology may result in progressive failure of the Fontan circulation. Importantly, high PVR is a strong predictor of mortality (Griffiths et al., Ann. Thorac. Surg. (2009), 88, 558-563).

Compositions of this invention may be administered in any of the foregoing compositions and according to dosage regimens established in the art whenever treatment of pulmonary hypertension, or of pulmonary vascular disease and/or cardiac dysfunction in functional single ventricular heart disease patients (especially in Fontan-palliated patients), is required.

Optimal dosages to be administered may be readily determined by those skilled in the art, and will vary with the strength of the preparation and the advancement of the disease condition. In addition, factors associated with the particular patient being treated, including patient age, weight, diet and time of administration, will result in the need to adjust dosages.

One skilled in the art will further recognize that human clinical trials including first-in-human, dose ranging and efficacy trials, in healthy patients and/or those suffering from a given disorder, may be completed according to methods well known in the clinical and medical arts.

### Definitions

As used herein, the following terms are defined with the following meanings, unless explicitly stated otherwise.

The term “about” when used before a numerical designation, e.g., pH, temperature, quantity, concentration and molecular weight, including range, indicates approximations which may vary by ±10%, ±5%, ±1%, or ±0.1%.

As used in the specifications and claims, the singular form “a”, “an” and “the” include plural references unless the context clearly dictates otherwise. For example, the term “a pharmaceutically acceptable carrier” may include a plurality of pharmaceutically acceptable carriers, including mixtures thereof.

The term “and/or” is intended to mean either or both of two components of the invention.

The term “subject,” “individual” or “patient’ is used interchangeably herein, refers to an animal, preferably a mammal, most preferably a human, who has been the object of treatment, observation or experiment. Preferably, the subject has experienced and/or exhibited at least one symptom of the disease or disorder to be treated and/or prevented.

The term “in need of treatment” and the term “in need thereof” when referring to treatment are used interchangeably and refer to a judgment made by a caregiver, e.g. physician, nurse, nurse practitioner, that a patient will benefit from treatment.

The term “pharmaceutically acceptable,” as used herein, refers to a component of a pharmaceutical composition that is compatible with the other ingredients of the formulation and not overly deleterious to the recipient thereof.

The term “therapeutically effective amount” as used herein, refers to the amount of active compound or pharmaceutical agent that elicits the biological or medicinal response in a tissue, system, or individual that is being sought by a researcher, healthcare provider or individual.

The term “w/w” as used herein, is intended to refer to mass fraction, i.e., the mass of a component divided by total mass of the whole. The term “% w/w” is intended to refer to the mass fraction multiplied by 100. Similarly, the term “w/v” refers to volume concentration, i.e., the mass of a component divided by total volume of the whole and the term “% w/v” refers to the volume concentration multiplied by 100.

The term “API” (Active Pharmaceutical Ingredient) as used herein, refers to a component in a therapeutic medication or nutraceutical substance that is biologically active.

The term “unit dose” refers to a single drug delivery entity, e.g., a tablet, capsule, dry powder, solution, dispersion etc., that is administered to an individual. The amount administered may vary according to numerous factors, including, e.g., the age of the individual, the weight of the individual, the genetic makeup of the individual, and the severity of symptoms exhibited by the individual to whom the drug is administered.

The unit dosage form (powder, granulation, tablet, sphere, or capsule) may be packaged into a blister foil pack, a stick pack, a sachet, a pouch, a bottle, or any other self-contained unit.

The term “excipient” as used herein is intended to mean components of a drug formulation other than the API that are added to a drug formulation to perform a specific function in the finished drug product. The excipient may aid in dissolution or dispersion of the API, improve the taste profile of the drug product among other things. An excipient composition is intended to refer to a combination of a plurality of excipients that can be added to an API to produce a finished drug product.

The term “composition” as used herein, is intended to encompass a product comprising the specified ingredients in the specified amounts, as well as any product which results, directly or indirectly, from combinations of the specified ingredients in the specified amounts.

The term “dispersible tablet”, as used herein, is intended to mean a tablet which, when submitted to the disintegration method according to the European Pharmacopoeia version 10.4, disintegrates completely in water at 15-22° C. in not more than 5 minutes, preferably less than 4 minutes or 3 minutes, and more preferably less than 2 minutes or even less than 1 minute.

The term “D10”, as used herein, is intended to refer to a particle size distribution that, when measured according to the method entitled “Determination of particle size distribution by laser diffraction” described in the “Methods” section below, is such that at least 10% of the particles have a particle size lower than the D10 value mentioned. The term “D50”, as used herein, is intended to refer to a particle size distribution that, when measured according to the method entitled “Determination of particle size distribution by laser diffraction” described in the “Methods” section below, is such that at least 50% of the particles have a particle size lower than the D50 value mentioned. The term “D90”, as used herein, is intended to refer to a particle size distribution that, when measured according to the method entitled “Determination of particle size distribution by laser diffraction” described in the “Methods” section below, is such that at least 90% of the particles have a particle size lower than the D90 value mentioned.

The term “mannitol” as used herein refers to D-mannitol. Thus, “β-Mannitol”, “δ-mannitol”, “Beta-Mannitol” and “Delta-Mannitol” refer to the corresponding solid forms of D-mannitol.

The term “hardness” as used herein refers to tablet hardness or tablet breaking force as described in the European Pharmacopoeia version 10.4, and can be used as a measure of the cohesiveness of the ingredients of a tablet.

Examples of suitable pharmaceutical compositions and dispersible tablets are provided in the detailed descriptions which follow herein. One skilled in the art will recognize that the listing of said examples is not intended, and should not be construed, as limiting in any way the invention set forth in the claims which follow thereafter.

Abbreviations used in the specification, particularly the Schemes and Examples, are as listed in Table A, below.

TABLE A
Abbreviations

| | |
|---|---|
| API | Active Pharmaceutical Ingredient |
| BU = | Blend Uniformity |
| CU = | Content Uniformity |
| DC = | Direct Compression |
| LOQ = | Limit Of Quantification |
| MgSt = | Magnesium Stearate |
| PAH = | Pulmonary Arterial Hypertension |
| WG = | Wet Granulation |

Compositions of macitentan of the present invention may be formulated according to the general schemes described below.

Process for the Preparation of Macitentan Dispersible Tablets:

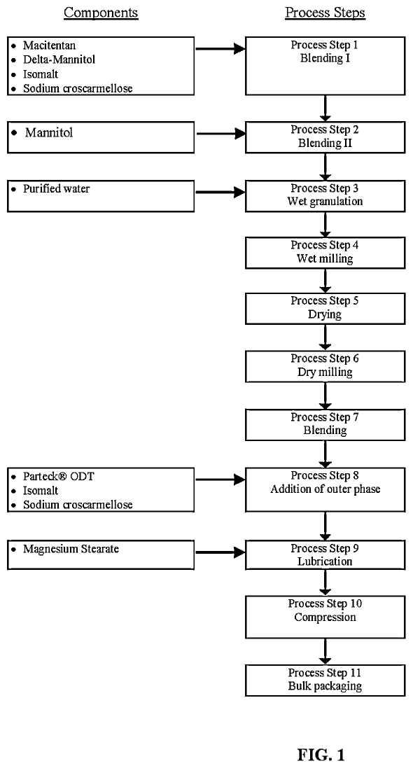

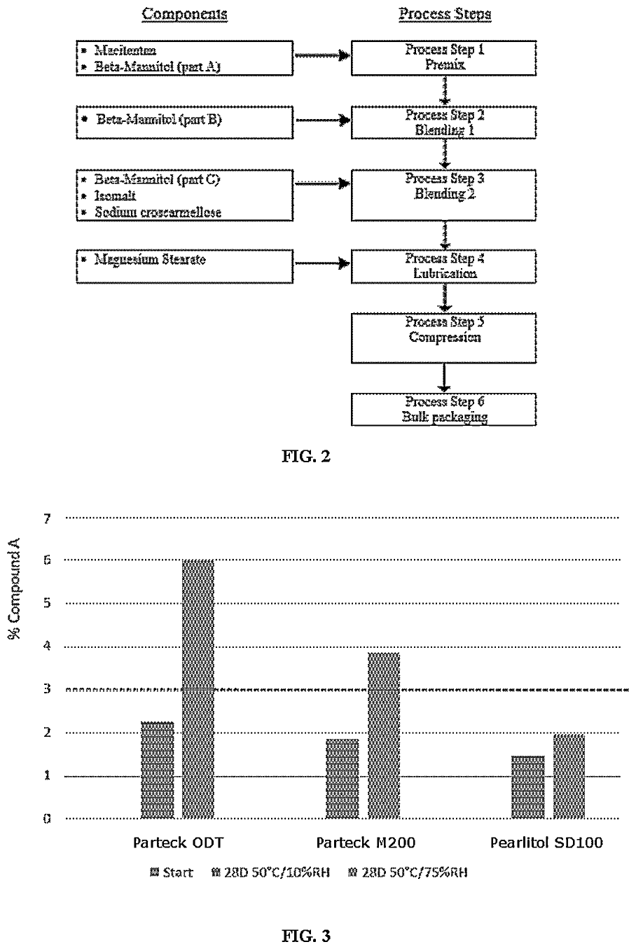

The pharmaceutical compositions of Examples 1-4 were prepared according to a processes summarized by the flow charts shown in FIG. 1 (wet granulation) and FIG. 2 (direct compression) accordingly. Any modifications from the flow charts are described in detail the respective Examples.

The following Examples are set forth to aid in the understanding of the invention, and are not intended and should not be construed to limit in any way the invention set forth in the claims which follow thereafter.

Aspects of the present teachings may be further understood in light of the following examples, which should not be construed as limiting the scope of the present teachings in any way.

### Example 1

Macitentan Dispersible Tablet Manufacturing Using DC with β-Mannitol

Direct compression trials were performed comparing 3 grades of β-mannitol: Parteck® M200, Pearlitol® SD100 and Parteck® ODT. These granular and spray-dried forms β-mannitol grades are on the market claiming improved compressibility and fluidity as compared to standard crystalline β-mannitol. Since Parteck® ODT is a combination product of β-mannitol and sodium croscarmellose, the formula of batch 3 was adjusted to align the actual mannitol and sodium croscarmellose content with the other batches.

TABLE 2
Grades of β-mannitol

| | Particle size | | |
|---|---|---|---|
| Mannitol grade | distribution | Specific surface area | |
| Parteck ® M200 | D50 142-231 μm | 3.0 | m²/g [Merck] |
| Pearlitol ® SD100 | D10-50-90: | 1.4 | m²/g [PreTaP] |
| | 64-111-165 μm | 0.6 | m²/g [Roquette] |
| Parteck ® ODT | D50 70-120 μm | 2.4-3.5 | m²/g [Merck] |

Formulations

TABLE 3
Tablet formulations with different grades of β-mannitol

| | | Amount per unit(mg) | | |
|---|---|---|---|---|
| Material name: | Function | Tablet 1 | Tablet 2 | Tablet 3 |
| Macitentan | Active | 0.5 | 0.5 | 0.5 |
| Parteck ® M200 | Filler | 38.4 | — | — |
| Pearlitol ® | Filler | — | 38.4 | — |
| SD100 | | | | |
| Parteck ® ODT | Filler | — | — | 40.6 |
| Isomalt | Compressibility | 5.0 | 5.0 | 5.0 |
| (GalenIQ ® 721) | enhancer | | | |
| Croscarmellose | Disintegrant | 5.60 | 5.6 | 3.4 |
| sodium | | | | |
| (AC-DI- | | | | |
| SOL) | | | | |
| Magnesium | Lubricant | 0.5 | 0.5 | 0.5 |
| stearate | | | | |
| Total | | 50.0 | 50.0 | 50.0 |

Manufacturing Procedure

Three batches of 6.4 kg scale of 0.5 mg strength were manufactured following an identical multi-step blending process. A geometrical blending approach was adopted for optimal blending efficiency. Macitentan (64 g) was combined with mannitol (150 g) by Turbula® blending (20 min). Mannitol (250 g) was added by blending another 20 min Turbula® blending. The resulting premix was sieved (0.5 mm) in a bin containing mannitol (0.5 kg) followed by bin blending (35 min). The API:mannitol blend was sieved bin-to-bin (0.5 mm) followed by further blending (35 min). Mannitol (1 kg) was added and blended (35 min). The remaining mannitol, isomalt and sodium croscarmellose were added and blended (35 min). Finally, magnesium stearate was added by bin blending for 2 min. The resultant mixture was then compressed into 5 mm round tablets, at target hardness of 20N.

Results

Blend flowability was slightly superior for the batch using Parteck® M200 (1.7 s/100 g) as compared to the batches with Pearlitol® SD100 (2.4 s/100 g) and Parteck® ODT (2.1 s/100 g) which can be attributed to the larger particle size of Parteck® M200. But all values can be considered decent. Further results of interest are summarized in Table 4. Batch homogeneity is best for Pearlitol® SD100, closely followed by Parteck® ODT. Both perform significantly better than Parteck® M200. Tablet parameters are similar, except for a shorter disintegration time for Parteck ODT. Yet, disintegration times are all <1 minute (limit is 3 minutes). The most prominent difference can be noted after exposing the tablets to stressed conditions. After 28 days at 50° C. 75% RH (the Compound A content has risen to 2% for Pearlitol® SD100, 4% for Parteck® M200 and even 6% for Parteck® ODT. Assay, purity and dissolution were similar across the batches (results not shown).

TABLE 4
Tablet parameters of batch formulations comprising
different grades of β-mannitol.

| | | | |
|---|---|---|---|
| | Parteck ® | Pearlitol ® | Parteck |
| | M200 | SD100 | ODT |
| Parameter | Tablet 1 | Tablet 2 | Tablet 3 |

Influence of mannitol grade on batch homogeneity

| | | | |
|---|---|---|---|
| BU, % RSD | 4.1% | 2.8% | 1.7% |
| CU, % RSD | 1.8% | 1.5% | 1.5% |
| CU weight corrected, % | 1.2% | 0.9% | 1.4% |
| RSD | | | |

Influence of mannitol on tablet parameters

| Compression force | 3.0 | kN | 3.0 | kN | 2.0 | kN |
|---|---|---|---|---|---|---|
| Tablet hardness | 38N | | 33N | | 33N | |
| Friability | 0.3% | | 0.3% | | 0.2% | |
| Disintegration time | 47 | s | 53 | s | 30 | s |

| | | | |
|---|---|---|---|
| Tablet weight variation | 1.3% | 1.1% | 0.7% |

Influence of mannitol on chemical stability

| | | | |
|---|---|---|---|
| % Compound A | 3.9% | 2.0% | 6.0% |
| (28 days at 50° C. | | | |
| 75% RH) | | | |

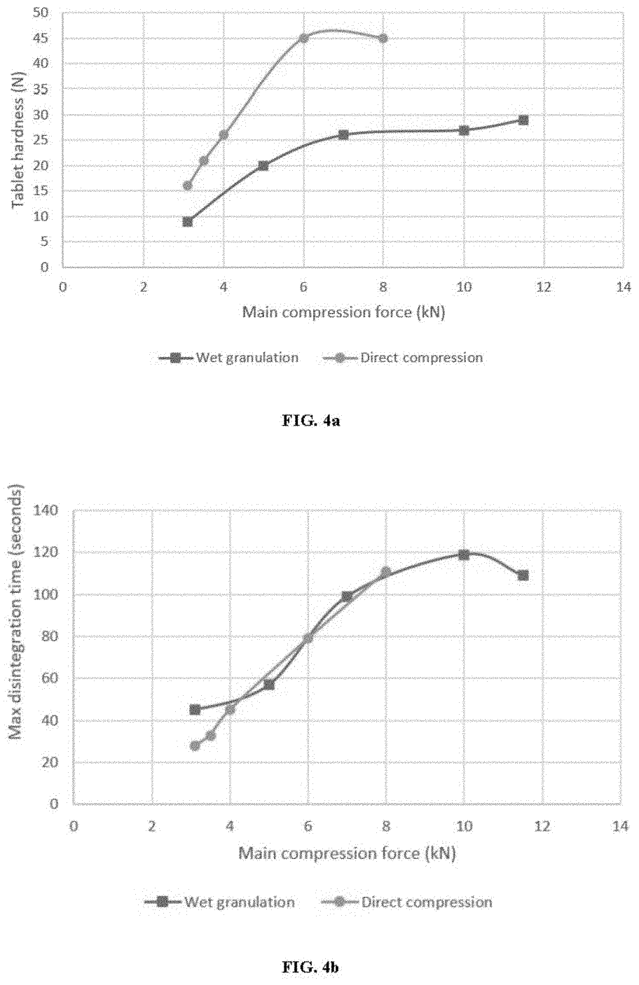

The stability data for the different batches is graphically illustrated in FIG. 3. FIG. 3 shows the amount of Compound A (hydrolysis degradation product of macitentan) with the tablets after storage conditions 50° C./10% relative humidity (RH) and 50° C./75% RH in comparison with the initial profile.

In conclusion, above results indicate DC (using Pearlitol® SD100) proves to be a promising alternative to WG (using δ-mannitol) for the preparation of macitentan dispersible tablets showing good batch homogeneity and acceptable tablet parameters and chemical stability.

### Example 2

Comparison of Direct Compression Formulation Over Wet Granulation Formulations

This experiment was performed to compare high shear wet granulation and direct compression processes and formulations to produce macitentan dispersible tablets on commercial production equipment. The DC formula (β-mannitol) and process show similar flowability, BU, tablet weight variation, friability, assay and dissolution rate as compared to the wet granulation formula (δ-mannitol) and process. The DC formula and process has advantage over wet granulation in terms of simplicity of formula and manufacturing process, excipient supply, compatibility of the blend and superior disintegration time. Overall, the process robustness of the novel formulation is superior to that formed via wet granulation.

Formulations

TABLE 5
Tablet formulations amounts for wet
granulation and direct compression

Tablet formula (mg/unit)

| | Wet | Direct |
|---|---|---|
| | granulation | compression |
| Inner phase | | |
| Macitentan | 0.5 | — |
| Delta-mannitol (Parteck delta M) | 29.5 | — |
| Croscarmellose sodium | 2.5 | — |
| Isomalt GalenIQ 800 | 2.5 | — |
| Outer phase | | |
| Macitentan | — | 0.5 |
| mannitol (Pearlitol SD100) | 9.0 | 38.4 |
| Isomalt GalenIQ 721 | 2.5 | 5.0 |
| Croscarmellose sodium | 3.0 | 5.6 |
| Magnesium stearate | 0.5 | 0.5 |
| SUM | 50.0 | 50.0 |

Manufacturing Procedure (Wet Granulation)

One batch of Macitentan 0.5 mg dispersible tablets using high shear granulation was produced at a batch size of 22.5 kg. Macitentan (0.225 kg), O-mannitol (13.28 kg), croscarmellose sodium (1.13 kg) and isomalt (1.13 kg) were blended in high shear mixer (10 min) and subsequently granulated with purified water (3.15 kg). After passing through co-mill (9.5 mm screen) wet granules were dried in a fluid bed dryer at 70° C. until a loss on drying of approx. 2% was reached. Dried granules were screened (1.0 mm) and blended with mannitol (4.05 kg), isomalt (1.13 kg) and croscarmellose sodium (1.35 kg) in bin blender (20 min). Finally, magnesium stearate was added by bin blending for 2 min. The resultant mixture was then compressed into 5 mm round tablets, at target hardness of 20N.

Manufacturing Procedure (Direct Compression)

One batch of Macitentan 0.5 mg dispersible tablets using DC was produced at a batch size of 15 kg. Macitentan (150 g) was combined with mannitol (350 g) by Turbula® blending (20 min). The resulting premix added to a bin containing mannitol (7 kg). The Turbula® contained was rinsed with mannitol (0.5 kg) which was subsequently added to the bin followed by bin blending (35 min). The API:mannitol blend was sieved bin-to-bin (1 mm) followed by further blending (35 min). The remaining mannitol, isomalt and sodium croscarmellose were added and blended (35 min). Finally, magnesium stearate was added by bin blending for 2 min. The resultant mixture was then compressed into 5 mm round tablets, at target hardness of 20N.

Results

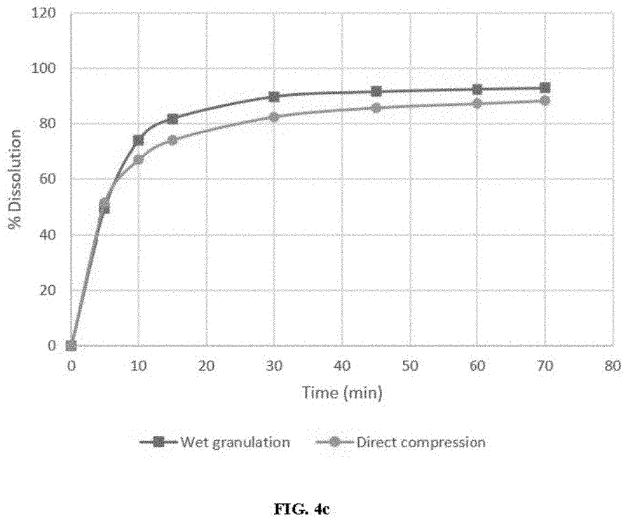

Flowability, tablet weight variation and BU of both batches are in line, indicating similar processability and homogeneity can be obtained with both formulations and manufacturing processes. A clear difference can however be observed in compactibility of both blends. The compression force-hardness profiles generated (See FIG. 4a) show that higher tablet hardness is achieved at lower compression forces for the DC formula. This elevated tablet hardness however does not affect the disintegration time of the tablets (See FIG. 4b). Macitentan tablets are dispersible tablets which implicates a compendial disintegration time limit of only 3 minutes. Hence, the balance between tablet hardness and disintegration time is a challenge in dispersible tablet compression. Sufficient tablet hardness is required to minimize tablet friability and avoid tablet defects during handling, shipment or packaging. On the other hand, there is a risk of disintegration time failure when tablet hardness is increased. Therefore, a superior hardness-disintegration time profile is beneficial to process robustness. Disintegration times of the 0.5 mg tablets (50 mg tablet weight) of both batches are well below the 3-minute limit yet the difference will become more pronounced at higher tablet weight. At 750 mg tablet weight and a comparable disintegration time of approx. 2 minutes the tablet hardness of the DC formula is 143N as compared to only 80N for wet granulation. The difference between both processes is even more pronounced when the dispersion time of the tablets on a spoon is compared. Patients are instructed to administer the drug product dispersed in a little water on a spoon. Tablets produced by DC disperse within 1 minute on a teaspoon with 3 ml water, whereas for the wet granulation tablets full dispersion takes about 4 minutes under the same conditions. Limited differences were observed with respect to assay, purity, CU, BU and dissolution (See FIG. 4c).

TABLE 6
Blend and tablet parameters for wet granulation
and direct compression batches.

| | | |
|---|---|---|
| Parameter | Wet granulation | DC |

Final

| | | |
|---|---|---|
| Flowability (s/100 g) | 1.9 | 2.2 |
| BU, RSD (%) | 2.5 | 2.0 |

Table

| | | |
|---|---|---|
| Pre-Compression force | 1.0 | 0.0 |
| Compression force (kN) | 5.0 | 3.5 |
| Weight variation (%) | 1.17 | 0.96 |
| Hardness (N) | 17 | 21 |
| Friability (%) | 0.4 | 0.3 |
| Disintegration time (s) | 63 | 27 |
| Dispersion on a spoon | 4 | 1 |
| CU, RSD (%) | 1.8 | 1.7 |
| Assay (%) | 96.4 | 97.3 |
| Purity (Compound A) (%) | 0.11 | <LOQ |

FIGS. 4a-4c depict results from the comparison of the wet granulation and direct compression formulations. Based on these results there are no major benefits from the use of δ-mannitol/wet granulation. By smart selection of the β-mannitol grade in combination with DC process, the product robustness can be enhanced while delivering drug product with comparable quality attributes

The results indicate direct compression using Pearlitol® SD100 proves to be a suitable alternative to wet granulation for the preparation of macitentan dispersible tablet showing good batch homogeneity and acceptable tablet parameters and chemical stability.

### Example 3

Optimization of Novel Macitentan Formula (Lubricant Level)

During manufacturing of macitentan dispersible tablets of 2.5 mg and 3.5 mg dose strengths comprising 1% magnesium stearate, picking defects in tablet inscriptions were observed. After extensive investigation, the problem was resolved without major impact on the tablet characteristics by increasing the magnesium stearate content in the composition up to 3%. Analytical data proved the magnesium stearate increase did not impact the dissolution rate of the macitentan dispersible tablets significantly.

Formulations

TABLE 7
Macitentan 2.5 mg dispersible tablet formulation.

| | Macitentan 2.5 mg dispersible tablets | | | | | |
|---|---|---|---|---|---|---|
| | 1% MgSt | | 2% MgSt | | 3% MgSt | |
| Material name: | mg | % | mg | % | mg | % |
| Macitentan | 2.50 | 1.0 | 2.50 | 1.0 | 2.50 | 1.0 |
| Mannitol | 192.00 | 76.8 | 189.50 | 75.8 | 187.00 | 74.8 |
| (Pearlitol ® | | | | | | |
| 100 SD) | | | | | | |
| Isomalt | 25.00 | 10.0 | 25.00 | 10.0 | 25.00 | 10.0 |
| (GalenIQ ® 721) | | | | | | |
| Croscarmellose | 28.00 | 11.2 | 28.00 | 11.2 | 28.00 | 11.2 |
| sodium | | | | | | |
| (AC-DI-SOL) | | | | | | |
| Magnesium stearate | 2.50 | 1.0 | 5.00 | 2.0 | 7.50 | 3.0 |

Manufacturing Procedure

Three batches of Macitentan 2.5 mg dispersible tablets using DC were produced at a batch size of 6.4 kg. Macitentan (64 g) was combined with mannitol (200 g) by Turbula® blending (20 min). The resulting premix added to a bin containing mannitol (2 kg). The Turbula® contained was rinsed with mannitol (0.5 kg) which was subsequently added to the bin followed by bin blending (15 min). The remaining mannitol, isomalt and sodium croscarmellose were added and blended (15 min). Finally, magnesium stearate was added by bin blending for 2 min. The resultant mixture was then compressed into 9 mm round tablets, at target hardness of 40N.

Results

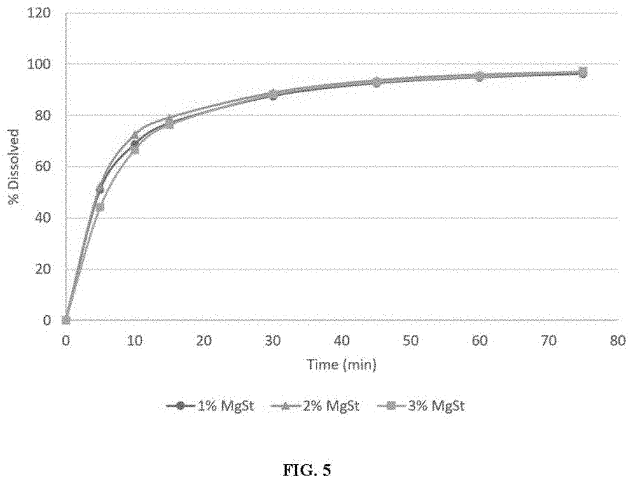

To investigate whether the level of lubricant in the formula (1%) was sufficient, Macitentan 2.5 mg dispersible tablet batches with increased magnesium stearate level (2% and 3%) were manufactured. There was a definite improvement with increased magnesium stearate concentration eliminating picking in tablets completely at a level of 3%. The increase in magnesium stearate did not impact tablet weight variation, hardness, friability or disintegration time nor did it impact on the dissolution the macitentan dispersible tablets (FIG. 5). Furthermore, no impact on BU, CU, assay and purity was observed (results not shown).

TABLE 8
Tablet parameters of formulations comprising
different Magnesium Stearate amounts.

| | 1% Magnesium | 2% Magnesium | 3% Magnesium |
|---|---|---|---|
| Parameter | stearate | stearate | stearate |
| Pre-Compression force | 1.0 | 1.0 | 1.0 |
| [kN] | | | |
| Compression force | 5.6 | 5.5 | 6.5 |
| [kN] | | | |
| Tablet appearance | Sticking | Light | No |
| | defects | sticking | sticking |
| | | defects | defects |
| Weight variation (%) | 2.0 | 1.4 | 1.9 |
| Hardness (N) | 43 | 39 | 46 |
| Friability (%) | 0.3 | 0.3 | 0.3 |
| Disintegration time (s) | 106 | 90 | 88 |

### Example 4

Macitentan Dispersible Tablets of 1 mg, 2.5 mg and 3.5 mg Dose Strengths

Macitentan dispersible tablets of 1 mg, 2.5 mg and 3.5 mg dose strengths, comprising 3% magnesium stearate, were manufactured to confirm formulation and manufacturing process for different strengths.

Formulations

TABLE 9
Macitentan 1.0 mg, 2.5 mg and 3.5 mg
dispersible tablets formulations.

Macitentan dispersible tablets

mg

| | | 1 mg | 2.5 mg | 3.5 mg |
|---|---|---|---|---|
| Material name | % | dose | dose | dose |
| Macitentan | 1.0 | 1.0 | 2.5 | 3.5 |
| Mannitol (Pearlitol 100 | 75.0 | 75.0 | 187.5 | 262.5 |
| SD) | | | | |
| Isomalt (GalenIQ 721) | 10.0 | 10.0 | 25.0 | 35 |
| Croscarmellose sodium | 11.0 | 11.0 | 27.5 | 38.5 |
| (AC-DI-SOL) | | | | |
| Magnesium stearate | 3.0 | 3.0 | 7.5 | 10.5 |

Manufacturing Procedure

Three batches of Macitentan dispersible tablets using DC were produced at a batch size of 15 kg. Macitentan (150 g) was combined with mannitol (350 g) by Turbula® blending (20 min). The resulting premix added to a bin containing mannitol (7 kg). The Turbula® container was rinsed with mannitol (0.5 kg) which was subsequently added to the bin followed by bin blending (45 min). The remaining mannitol, isomalt and sodium croscarmellose were added and blended (20 min). Finally, magnesium stearate was added by bin blending for 2 min. The resultant mixture was then placed on a rotary tablet press. Different punch sets were used to allow for differentiation across the strengths.

Results

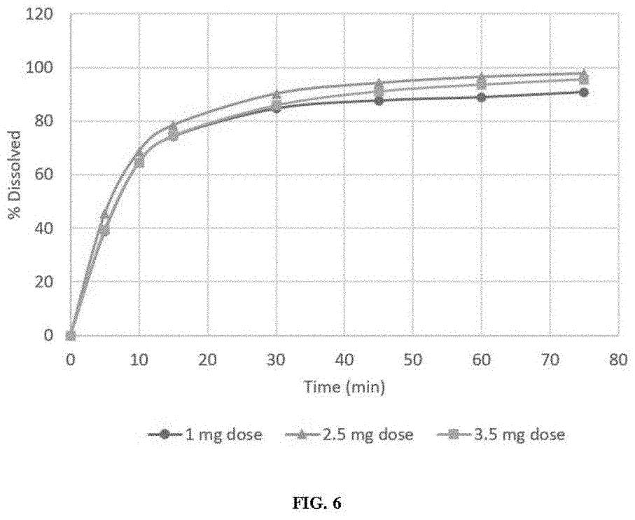

Macitentan dispersible tablets share a common blend which is compressed dose proportionally to obtain different tablet strengths. Blend homogeneity is passing the acceptance criterium of SD≤3.0% very well and shows little inter-batch variability. Tablet parameters and content uniformity were good for the different tablet sizes and shapes. Similar dissolution profiles were obtained across the different strengths (FIG. 6).

TABLE 10
Blend and tablet parameters for Macitentan dispersible
tablets of 1.0, 2.5 and 3.5 mg dose strength.

| Parameter | 1.0 mg Dose | 2.5 mg Dose | 3.5 mg Dose |
|---|---|---|---|
| BU, SD (%) | 1.4 | 1.0 | 0.7 |
| Punch | Oval, | Round, 9 mm | Oval, |
| | 8 × 5 mm | | 9 × 5.5 mm |
| Pre-Compression force | 3 | 1 | 2 |
| [kN] | | | |
| Compression force | 7 | 7 | 12 |
| [kN] | | | |
| Tablet appearance | Ok | Ok* | Ok |
| Weight variation (%) | 0.85 | 0.70 | 0.64 |
| Hardness (N) | 50 | 43 | 80 |
| Friability (%) | 0.2 | 0.2 | 0.1 |
| Disintegration time (s) | 64 | 65 | 104 |
| CU, RSD (%) | 1.0 | 1.0 | 1.6 |

*About 1% tablets with small edge chipping defects observed yet this was resolved by increasing the hardness for subsequent batches.

Methods

The tests for flowability, tablet hardness, friability, disintegration time were conducted as described in the European Pharmacopoeia version 10.4.

Flowability: ISO Lab flow funnel, method according to European Pharmacopoeia version 10.4

Tablet hardness: Sotax® HT1 hardness tester (Experiments 1 and 3), Schleuniger 8M® (experiment 2)

Friability: Sotax® FT2 friabilator, method according to European Pharmacopoeia version 10.4, 2.9.7. Friability of Uncoated Tablets)

Disintegration time: Sotax® DT2 disintegration tester, method according to European Pharmacopoeia version 10.4, Monograph Tablets (0478) for dispersible tablets (Dispersible tablets disintegrate within 3 minutes when examined by 5.3 Disintegration test for tablets and capsules, but using water R at 15-25° C.)

Disintegration on a spoon: A tablet is put on a tablespoon and 3 ml water is added. Dispersion is examined by gently touching the tablet mass with a spatula. Full dispersion is achieved when no hard mass is left.

Assay/Purity

The HPLC conditions for measuring the assay, purity and tablet content data in FIG. 3 and Tables 4, 6, and 10 were as follows:

Diluent:

Solution A: dissolve 1.6 g ammonium bicarbonate in 1000 ml de-ionized water and bring to pH 9 with ammonium hydroxide solution

Solution B: acetonitrile

Combine 500 ml of solution A with 500 ml of solution B and mix

Mobile Phases:

Mobile phase A: Acetonitrile/di-ionized Water/Trifluoroacetic, 500/500/5 ml

Mobile phase B: Acetonitrile/di-ionized Water/Trifluoroacetic acid, 650/350/5 ml

Operating Parameters:

TABLE 11
Gradient Program

| Time | Mobile Phase A | Mobile Phase B | Flow |
|---|---|---|---|
| (Minutes) | (% vol.) | (% vol.) | (ml/min) |
| 0 | 100 | 0 | 1.0 |
| 12 | 100 | 0 | 1.0 |
| 42 | 65 | 35 | 1.0 |
| 42.1 | 100 | 0 | 1.0 |
| 50 | 100 | 0 | 1.0 |

Column: Nucleosil® C18 HD, EC 250/4 100-5, 250 mm length×4.0 mm i.d., 5 μm particle size
Detection: UV
Wavelength: 260 nm, 4 nm
Column Temperature: 25° C.
Auto-Sampler Temperature: 5° C.
Analysis Run Time: 50 minutes
Injection volume: 80 μl (injected amount=4 μg)
Retention time macitentan: 21 min
Relative retention time Compound A: 0.22 (reference: Macitentan)

Dissolution Test

The analytical method used to obtain dissolution test results in FIGS. 4c, 5 and 6 is summarized in Table 12:

TABLE 12
Dissolution Testing Parameters

| Parameter | Value |
|---|---|
| Apparatus | Paddle (USP type 2, Ph. Eur. JP) |
| Dissolution | 0.05M sodium phosphate buffer pH 6.8 with |
| medium | 0.5% cremophor A25 |
| Medium | 37.0 ± 0.5° C. |
| temperature | |
| Medium volume | 500 ml (0.5 mg dose strength) |
| | 900 ml (other dose strengths) |
| Paddle rotation | 50 rpm |
| speed | |
| Sampling time | 5, 10, 15, 30, 45, 60 min |
| points | |
| Sample filter | Glass fiber (e.g. Gelman, 1 μm) |
| Analytical finish | HPLC with UV detection at 260 nm |

The HPLC conditions for measuring the macitentan content in the samples were as follows:

Diluent

Solution A: dissolve 1.6 g ammonium bicarbonate in 1000 ml de-ionized water and bring to pH 9 with ammonium hydroxide solution

Solution B: acetonitrile

Combine 500 ml of solution A with 500 ml of solution B, mix well and degas before use.

Mobile Phase:

Mobile phase A: Combine 850 ml acetonitrile, 150 ml water and 5 ml trifluoroacetic acid, mix well and degas before use

Chromatographic Conditions:
Column: EC 250/3 Nucleodur® C18 gravity 3 μm, 250 mm length×3.00 mm i.d., 3 μm particle size
Detection: UV
Wavelength: 260 nm
Column Temperature: 25° C.
Auto-Sampler Temperature: 5° C.
Mobile phase: Isocratic mode
Flow rate: 0.5 ml/min
Analysis Run Time: 10 minutes
Injection volume: 100 μl

Laser Diffraction Method for Determining Particle Size Distribution

The Particle Size Distribution of a sample is determined via dry dispersion laser diffraction by dry measurements performed on 10 g of powder using the apparatus Mastersizer 2000 (Malvern Instruments, Worcestershire, UK) and the Sirocco 2000 dry dispersion unit. The test is performed in triplicate and averages are calculated using the Malvern software. The applied instrument parameters for this method are described in Table 13 hereafter:

TABLE 13
Dry Dispersion Laser Diffraction Parameters

| Parameter | Value | |
|---|---|---|
| Instrument | Mastersizer 2000 (Malvern Instruments, | |
| | Worcestershire, UK) | |
| Method | Frauenhofer | |
| Models | General purpose; Normal | |
| Measurement time | 30 | s |
| Background time | 30 | s |
| Lower obscuration limit | 0.2 | |
| Upper obscuration limit | 25 | |
| Obscuration filter | 1 min time-out | |
| Sample tray | General purpose | |
| Vibration rate | 50% | |
| Dispersive air pressure | 2 | bar |

| | |
|---|---|
| Aliquots | 1 |

BET Method for Determining Specific Surface Area

The Specific Surface Area of a solid sample is tested using the method described using the standardized method ISO 9277:2010 thanks to the measurement of the amount of physically adsorbed gas according to the Brunauer, Emmett and Teller (BET) method. The adsorption analysis with nitrogen as adsorptive is recorded at 77 K on a Micromeritics® TriStar II 3020 surface area analyzer, with a relative pressure from p₀/p=0.01 to p₀/p=0.30. Prior to the actual adsorption investigations, the samples are pre-treated at 300° C. in vacuum for 16 hours. The dry sample mass obtained after that pre-treatment is used in the various calculations performed according to ISO 9277:2010.

### Example 5

Macitentan Dispersible Tablets of 2.5 mg Dose Strength: Formula Variation

Macitentan dispersible tablets of 2.5 mg dose strength with varying levels of disintegrant and lubricant were manufactured to confirm formulation robustness. For Tablet 2 the croscarmellose sodium level was reduced by 50%, resulting in a croscarmellose sodium concentration of 5.5%. For Tablet batch 3 the magnesium stearate level was increased to 5% of the formulation. These differences in formulation were compensated by changes in mannitol amount so that the resulting tablet weight remained at 250 mg.

TABLE 14
2.5 mg Tablet Formulations

| | | Amount per unit(mg) | | |
|---|---|---|---|---|
| | | | Tablet 2 | Tablet 3 |
| Material | | Tablet 1 | Reduction | Increased |
| name: | Function | Reference | disintegrant | lubricant |
| Macitentan | Active | 2.50 | 2.50 | 2.50 |
| Beta-mannitol | Filler | 187.50 | 201.25 | 182.50 |
| (Pearlitol ® | | | | |
| SD100) | | | | |
| Isomalt | Filler- | 25.00 | 25.00 | 25.00 |
| (GalenIQ ® 721) | Compressibility | | | |
| | enhancer | | | |
| Croscarmellose | Disintegrant | 27.50 | 13.75 | 27.50 |
| sodium | | | | |
| (AC-DI-SOL ®) | | | | |
| Magnesium | Lubricant | 7.50 | 7.50 | 12.50 |
| stearate | | | | |
| Total | | 250.00 | 250.00 | 250.00 |

Manufacturing Procedure

Three batches of Macitentan dispersible tablets using DC were produced at a batch size of 1.5 kg. Macitentan (15 g) was combined with mannitol (35 g) by Turbula® blending (20 min). The resulting premix added to a bin containing mannitol (0.7 kg). The Turbula® container was rinsed with mannitol (0.05 kg) which was subsequently added to the bin followed by bin blending (45 min). The remaining mannitol, isomalt and sodium croscarmellose were added and blended (20 min). Finally, magnesium stearate was added by bin blending for 4 min. The resultant mixture was then compressed on a rotary tablet press.

Results

Tablet parameters and content uniformity were good for the different tablet batches that were obtained, indicating the tablet formulation robustness.

TABLE 15

| | | Tablet 2 | Tablet 3 |
|---|---|---|---|
| | Tablet 1 | Reduction | Increased |
| Parameter | Reference | disintegrant | lubricant |
| Tablet appearance | Ok | Ok | Ok |
| Weight variation (%) | 1.06 | 0.65 | 1.25 |
| Hardness (N) | 72 | 70 | 67 |
| Friability (%) | 0.2 | 0.2 | 0.2 |

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

Throughout this application, various publications are cited. The disclosure of these publications is hereby incorporated by reference into this application to describe more fully the state of the art to which this invention pertains.

What is claimed is:

1. A dispersible tablet comprising:
0.9-1.1% w/w macitentan or a pharmaceutically acceptable salt, solvate, hydrate or morphological form thereof,
67.5-82.5% w/w β-mannitol,
9-11% w/w isomalt,
9.9-12.1% w/w croscarmellose sodium, and
2.7-3.3% w/w magnesium stearate.

2. The dispersible tablet of claim 1, wherein the macitentan is present in an amount of about 1 mg, about 2.5 mg, about 3.5 mg, or about 5 mg.

3. The dispersible tablet of claim 2, wherein the β-mannitol has a particle size distribution having a D10 value of from 10 to 60 μm, a D50 value of from 60 μm to 140 μm, and a D90 value of from 140 μm to 220 μm.

4. The dispersible tablet of claim 2, wherein the β-mannitol has a specific surface area of 0.5 to 1.5 m²/g.

5. The dispersible tablet of claim 3, having a hardness of 20-120 N.

6. The dispersible tablet of claim 1, comprising about 1% w/w macitentan, about 75% w/w β-mannitol, about 10% w/w isomalt, about 11% w/w croscarmellose sodium, and about 3% w/w magnesium stearate.

7. The dispersible tablet of claim 6, wherein the β-mannitol has a particle size distribution having a D10 value of from 10 to 60 μm, a D50 value of from 60 μm to 140 μm, and a D90 value of from 140 μm to 220 μm.

8. The dispersible tablet of claim 1, wherein the dispersible tablet is prepared by direct compression process.

9. A method of treating pulmonary hypertension, comprising administering to a patient in need thereof the dispersible tablet of claim 1.

10. The method of claim 9, wherein the pulmonary hypertension is pulmonary arterial hypertension.

11. A method of treating pulmonary hypertension, comprising administering to a patient in need thereof the dispersible tablet of claim 6.

12. The method of claim 11, wherein the pulmonary hypertension is pulmonary arterial hypertension.
